(19) United States
(12) Patent Application Publication
(10) Pub. No.: US 2021/0153848 A1
(43) Pub. Date: May 27, 2021

(54) SKIN CANCER TREATMENT

(71) Applicant: Imcomet B.V., Rotterdam (NL)

(72) Inventor: Alexandre Charles Roger Motta, Rotterdam (NL)

(21) Appl. No.: 16/690,487

(22) Filed: Nov. 21, 2019

(51) Int. Cl. A61B 10/00; C07K 16/28; A61K 33/243; A61K 31/555; A61K 31/495; A61K 31/337; A61K 31/655

(52) U.S. Cl. CPC A61B 10/0045; C07K 16/2818; C07K 16/2827; A61K 33/243; A61K 45/06; A61K 31/495; A61K 31/337; A61K 31/655; A61K 31/555

(57) ABSTRACT

The present invention relates to the fields of medicine and cancer, more particularly to the field of skin cancer (e.g. melanoma and non-melanoma cancers) and skin cancer treatment. Provided are methods for treating skin cancer using microneedle devices for the removal of an amount of interstitial fluid or tumor interstitial fluid from a skin tumor. Also provided are therapeutic compounds for use in the treatment of skin cancer, wherein the treatment comprises removal of an amount of interstitial fluid or tumor interstitial fluid from a skin tumor.

### FIELD OF THE INVENTION

The present invention relates to the fields of medicine and cancer, more particularly to the field of skin cancer (e.g. melanoma and non-melanoma cancers) and skin cancer treatment. Provided are methods for treating skin cancer using microneedle devices for the removal of interstitial fluid or tumor interstitial fluid from a skin tumor. Also provided are therapeutic compounds for use in the treatment of skin cancer, wherein the treatment comprises removal of interstitial fluid or tumor interstitial fluid from a skin tumor.

### BACKGROUND OF THE INVENTION

Skin cancer, such as melanoma and non-melanoma skin cancer (NMSC) (including basal-cell carcinoma, and squamous-cell carcinoma), represents one of the most common type of malignancy, particularly in the Caucasian population (white population) (e.g. see Whiteman et al (2016), J Invest., Dermatol. Vol: 136, pages 1161-71; Apalla et al (2017), Dermatol Ther (Heidelb), Vol: 7 (Suppl 1):S5_S19). The incidence rate of skin cancers (melanoma and NMSC) is increasing worldwide. For instance, according to the World Health Organization, between 2 and 3 million non-melanoma skin cancers and 132,000 melanoma skin cancers occur globally each year. One in every three cancers diagnosed is a skin cancer and, according to Skin Cancer Foundation Statistics, one in every five Americans will develop skin cancer in their lifetime (Beifus et al (2017), BMJ Open 2017; 7:e017196. doi: 10.1136/bmjopen-2017-017196).

The etiology of skin cancer is diverse and depends on the skin cancer type, particular population, genetic makeup, gender, skin tone, personal immune history and environmental factors, life stage, etc. One important risk factor or causal factor identified is exposure to ultraviolet radiation from sun exposure (e.g. see Narayanan et al (2010), International Journal of Dermatology, Vol: 49 (9), pages 978-86; Gordon et al (2013), Seminars in Oncology Nursing, Vol 29 (3), pages 160-169; Apalla et al (2017), Dermatol Ther (Heidelb), Vol: 7 (Suppl 1):S5-S19).

While the causes of skin cancers continue to be actively researched for the purpose of finding new cures or treatments, prevention strategies to prevent or reduce the incidence of skin cancers are being increasingly advocated and implemented in several countries. For instance, such strategies often consist of encouraging behavioral changes (e.g. limit sun exposure, use adequate sun protection (sunscreen), avoid bed tanning, etc.) and/or enhancing early detection.

Treatment options for skin cancers are diverse and dependent on the specific type of skin cancer, location of the skin cancer, age of the person, and whether the cancer is primary or a recurrence, etc. For instance, Mohs surgery may be best indicated for an infiltrating basal-cell carcinoma. Topical chemotherapy or immuno-modulatory agents might be indicated for large superficial basal-cell carcinoma for good cosmetic outcome, whereas it might be inadequate for nodular basal-cell carcinoma or invasive squamous-cell carcinoma. In general, melanoma is poorly responsive to radiation or chemotherapy. However, treatments for metastatic melanoma often include for instance, immunotherapy agents (e.g., immune checkpoint inhibitors) such as ipilimumab (anti-CTLA-4), pembrolizumab (anti-PD-1), and nivolumab (anti-PD-1); BRAF inhibitors, such as vemurafenib and dabrafenib; and MEK inhibitors such as trametinib, and other drug treatments.

However, pharmaceutical skin cancer treatments, such as those above are rarely curative, are often associated with side effects (toxicity) and/or are not therapeutically effective in all patients or cease to be therapeutically effective over time (development of drug resistance). In light of this, improved or alternative products, compositions, methods and uses thereof for treating skin cancer would be highly desirable. However, these are not yet readily available.

Therefore, there is a need in the art for reliable, efficient and reproducible methods for treating skin cancers (e.g. melanoma and non-melanoma) which are devoid of at least some of the limitations above and/or which can enhance the efficacy of conventional cancer treatments. Accordingly, the technical problem underlying the present invention can be seen as the provision of improved or alternative methods for treating skin cancer. The technical problem is solved by the embodiments characterized in the claims and herein below.

### BRIEF DESCRIPTION OF THE DRAWINGS

Embodiments of the invention are further described hereinafter with reference to the accompanying drawings, in which:

FIG. 1 depicts the impact of healthy and inflamed conditioned media on TNF alpha-induced inflammatory response (as measured by IL-8 levels), as carried out in example 1.

FIG. 2 depicts the impact of healthy and inflamed exosomes on TNF alpha-induced inflammatory response (as measured by IL-6 levels), as carried out in example 2.

### DETAILED DESCRIPTION

### Definitions

The section headings used herein are for organizational purposes only and are not to be construed as limiting the subject matter described.

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular forms “a,” “an” and “the” include plural referents unless the context clearly dictates otherwise. For example, a method for administrating a drug includes the administrating of a plurality of molecules (e.g. 10's, 100's, 1000's, 10's of thousands, 100's of thousands, millions, or more molecules).

As used herein, the term “and/or” indicates that one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, etc.

The term “to comprise” and its conjugations as used herein is used in its non-limiting sense to mean that items following the word are included, but items not specifically mentioned are not excluded. It also encompasses the more limiting “to consist of”.

The term “skin cancer (s)” or “skin tumor” as used herein refers to cancer(s) that originate from the skin's cells. Specifically, skin cancers are the results of the development of abnormal cells that have the ability to invade or spread to other parts of the body. Skin cancer or tumors are located on and/or within the skin. There are three main types of skin cancers: basal-cell skin cancer, squamous-cell skin cancer and melanoma. The first two are known as non-melanoma skin cancer (NMSC).

Basal-cell skin cancer (also known as basal cell carcinoma) can be defined as abnormal, uncontrolled growths or lesions that arise in the skin's basal cells, which line the deepest layer of the epidermis (the outermost layer of the skin). Basal-cell skin cancer often looks like open sores, red patches, pink growths, shiny bumps, or scars and are usually caused by a combination of cumulative and intense, occasional sun exposure. Basal-cell skin cancer almost never spreads (metastasizes) beyond the original tumor site but if they do, it may become life-threatening. This form of skin cancer is the least deadly and with proper treatment may be completely eliminated mostly by surgical removal which often leaves a scar, or by treatment with immune-modulating topical formulations, which induce severe local inflammatory reaction for the duration of the treatment (5-12 weeks). A rare but aggressive form of basal cell carcinoma is the Merkel cell carcinoma. This form of cancer is often deadly and can nowadays be treated by immunotherapy (Cameron et al J (2018), Am. Acad. Dermatol. Pii: S0190-9622 (18), 30775-8) doi: 10.1016/j.jaad.2018.03.060).

Squamous-cell skin cancer (also known as squamous cell carcinoma) is the second most common form of skin cancer, which develops as a result of uncontrolled growth of transformed cells arising from the squamous cells in the epidermis, the skin's outermost layer. Squamous-cell skin cancer often looks like scaly red patches, open sores, warts or elevated growths with a central depression, which may crust or bleed. They can become disfiguring and sometimes deadly if allowed to grow. This form of cancer can be metastatic and recurrent: i.e., it can reappear after removal by surgery, chemotherapy or radiation therapy. In these cases, immunotherapy can be applied (Potenza et al (2017), Bio. Res. Int., 9489163. doi: 10.1155/2018/9489163).

Basal cell and squamous cell carcinoma are also collectively referred to as “non-melanoma skin cancer” or NMSCs. Melanoma is the most dangerous form of skin cancer. Melanoma tumors originate in the pigment-producing melanocytes in the basal layer of the epidermis. Melanomas often resemble moles. The majority of melanomas are black or brown, but they can also be skin-colored, pink, red, purple, blue or white. Melanoma is caused mainly by intense, occasional UV exposure (frequently leading to sunburn), especially in those who are genetically predisposed to the disease. Warning signs of malignant melanoma include change in the size, shape, color or elevation of a mole. Other signs are the appearance of a new mole during adulthood or pain, itching, ulceration, redness around the site, or bleeding at the site (Helgadottir et al (2018), Front. Oncol., at https://doi.org/10.3389/fonc.2018.00202).

The term “subject” or “patient” (used interchangeably) as used herein preferably refers to a human subject male or female, adult, child or infant, suffering from a skin cancer (e.g. melanoma), regardless of the stage or state of the cancer. In some embodiments, the term “subject” or “patient” (used interchangeably) as used herein may refer to a non-human subject (e.g. pets such as cats, dogs, horses, or non-human primates, or other animals), which can be male or female, adult, juvenile, or infant, suffering from a skin cancer (e.g. melanoma), regardless of the stage or state of the cancer

The term “interstitium” or “interstitial space” as used herein is a commonly used term to refer to connective and supporting tissues in the body. The interstitial space is located outside the blood and lymph vessels and parenchymal cells and consists of two major phases: the interstitial fluid (IF) and the structural molecules comprising the extracellular matrix (ECM) (collectively known as stroma). It has been shown that, compared with normal interstitium, the tumor stroma comprises an abnormal number of immune cells, endothelial cells, and fibroblasts having a dysregulated biology, which are believed to provide support to tumor cells during the transition to malignancy (Emon et al (2018), Comput. Struct. Biotechnol. J., Vol. 16, pages 279-287). Moreover, the extracellular matrix (proteins that act as a scaffold for cell growth and migration) may be altered by cancer cells and may promote expansion and metastasis of the cancer. In addition, the tumor stroma is highly hypoxic due to the increased metabolic activity of the tumor cells. This renders the tumor microenvironment unfavorable for immune cells to mount a proper anti-tumor response (Wegiel et al (2018), Front. Oncol., at https://doi.org/10.3389/fonc.2018.00284).

The term “microenvironment” (ME) as used herein refers to the net effect of the multitude of signals that are exchanged at any time between all the cells of a given tissue. Technically speaking, the ME is defined by the composition of the extracellular matrix and interstitial fluid that provides key regulatory and developmental signals to the resident cells. Within a given organ or tissue, three main types of signals are delivered to tissue-resident and infiltrating cells via the ME: i) soluble factors produced by tissue-resident cells (cytokines, chemokines, lipid mediators, proteases and other enzymes, exosomes and micro vesicles that contain various types of biologically active molecules such proteins, lipids or RNAs), ii) physical and chemical interactions with the various components of the extracellular matrix or basal membranes (collagen, fibrinogen, laminin, a network of polymerized proteins on which chemokines and cytokines can bind), and iii) direct cell-cell contact (receptor-ligand type of interaction).

Under normal conditions, the tissue microenvironment (ME) promotes healthy homeostasis: cell renewal, proliferation and apoptosis as well as baseline metabolism and function. It also controls the development of a (micro) vascular network that is adapted to the local needs. In addition, it drives the migration and tissue context-specific differentiation of patrolling immune cells for immune surveillance and stem cells from their local niches for tissue renewal. In response to a challenge (injury, infection, or any type of dysfunction), the tissue ME orchestrates the resulting inflammatory reaction by creating an immune cell-attractant milieu and by providing survival and proliferation signals for infiltrating immune cells as well as “stop” signals when the dysfunction has been eliminated. Later, a healthy ME will orchestrate the recruitment and function of the stem cells involved in tissue repair to eventually re-establish homeostasis. In the case of skin cancer, the ME is highly immunosuppressive, thereby preventing the immune system to recognize and destroy the tumor. It also promotes tumor growth as well as the cancerogenic transformation of healthy surrounding cells and actively participates in metastasis. In addition, it drives the switch of surrounding fibroblasts into so-called cancer associated fibroblasts (CAFs). These CAFs have been shown to promote tumor growth, as well as immune cells exclusion (https://science.sciencemag.org/content/348/6230/74.long).

The term “interstitial fluid” (often abbreviated as “IF”) as used herein refers to the extracellular fluid that fills the spaces between most of the cells of the body and may provide a substantial portion of the liquid environment of the body. Essentially, the interstitial fluid serves as a reservoir and transportation system for nutrients and solutes and other molecules to organs, cells, and capillaries. It also contains an abundance of substances that are either produced locally or transported to the organ by the blood circulation. For instance, the interstitial fluid may comprise molecules or proteins such as chemokines, cytokines, enzymes, soluble extracellular matrix proteins, exosomes, extracellular vesicles and apoptotic bodies, lipid mediators and others. The composition and chemical properties of the interstitial fluid vary among organs and undergo changes in chemical composition during normal function, as well as during body growth, conditions of inflammation, and development of diseases (e.g. cancer such as skin cancer). The IF plays an important role in the communication between the different cells of a given tissue and, together with the stroma, defines the so-called tissue microenvironment (ME, as defined herein). In the case of a tumor, the interstitial fluid (often referred to as tumor interstitial fluid (TIF)) contains altered levels of soluble signaling molecules and vesicles that shape a specific tumor micro-environment (TME). The TME have been shown to promote tumor growth, impair the function of the local immune cells and/or prevent their infiltration in the tumor, as well as induce metastasis (Maman and Witz (2018), Nat. Rev. Cancer., Vol. 18 (6), pages 359-376). In addition the exosomes and extracellular vesicles produced by the tumor not only modify the physiology of the tissue-resident cells but can also enter the blood stream and prime for the metastatic cells that escape the tumor to attach and grow at distant sites in the body (the so-called pre-metastatic niche) (Li et al (2018), Int. J. Cancer, doi: 10.1002/ijc.31774; Lobb et al (2017), Semin. Cell Dev. Biol., doi: 10.1016/j.semcdb).

The term “microneedle device” as used herein refers to a device comprising one or more microneedles, preferably a plurality of microneedles, the later which are suitable for penetrating the skin deep enough so as to gain access to the interstitial fluid comprised within the skin tumor environment and remove or extract an amount of said interstitial fluid out of the skin tumor (cells) environment. Preferably, the microneedles penetrate the skin at a depth ranging between about 0.05 and 1.5 mm, such as for instance between about 0.1 to 1.4 mm, between about 0.2 to 1.3 mm, between about 0.3 and 1.2 mm, between about 0.4 and 1.1 mm, between about 0.5 and 1.0 mm, or between about 0.6 to 0.900 mm, or preferably between about 0.3 and 0.700 mm. It is understood that the depth can be adjusted depending on the cancer type (e.g. melanoma which are located deeper in the skin) and/or the stage of the cancer (e.g. advanced skin cancer which invade deeper layers of the skin).

For instance, in the case of advance skin cancer (e.g. melanoma), the microneedles may be adjusted to reach a deeper depth, e.g. up to about 1.5 mm.

It is further understood that the plurality of needles can be of varying lengths, such that a subgroup of needles is shorter or longer than another subgroup of needles, allowing for the extraction of ISF from various layers of the tumor.

In an embodiment, any microneedle device which are suitable for removing or extracting an amount of said interstitial fluid out of a skin tumor (cells) environment can be used in the method of the present invention. Non-limiting examples of suitable porous microneedle devices can be purchased for instance at MyLifeTechnologies (Leiden, NL). Suitable hollow microneedle device can be purchased for instance at Ascilion (Kista, Sweden). Non-limiting examples of microneedle device capable of removing or extracting an amount of said interstitial fluid include the microneedle device described in US2016296149 or the microneedle device described in Mukerjee et al (2004) Sensors and Actuators A., Vol. 114, pages 267-275).

The microneedle device as taught herein is preferably placed on or in close proximity of the skin cancer, covering the skin cancer completely or partly. In some embodiments, the microneedle device is positioned on the skin tumor, or on skin tissue adjacent to the skin tumor or in part on the skin tumor and in part on skin adjacent to the skin tumor.

The microneedle device as taught herein is preferably comfortable (do not cause pain or irritation or tension, etc.), minimally invasive (implanted in the superficial layer of the skin, e.g. a depth up to about 1.5 mm from the skin surface, e.g. between about 0.3 and 0.7 mm), are easy to apply and remove, stay in place for the desired duration of the treatment, and do not require preparation of the skin.

In an embodiment, the microneedle device having the desired characteristics may be produced by 3D printing technology. This would be advantageous to obtain a microneedle device that substantially fits or covers the shape and size of the skin tumor to be treated (which varies between patients but also within a same patient over time) or to make any other adjustments tailored to the cancer patient, e.g., such as adjusting the length of the needles, density of the needles per square cm, etc. Printers allowing the fabrication of such devices can be purchased from companies such as Nanoscribe (Eggenstein-Leopoldshafen, Germany).

The term “exosomes” or “extracellular vesicles (EVs)” as used herein refers to cell-derived vesicles that are present in eukaryotic fluids, including blood, urine, and cultured medium of cell cultures. Exosomes or EVs contain various molecular constituents of their cell of origin, including proteins, lipids and RNA. The exosomal or EV protein composition varies with the cell and tissue of origin, as well as the physiological status of the said cell or tissue (healthy, inflamed, cancerous etc.). Exosomes or EVs can transfer molecules from one cell to another via membrane vesicle trafficking, thereby influencing the immune system, such as dendritic cells, T, B or NK cells, and play a functional role in mediating innate and adaptive immune responses to pathogens and tumors.

The term “skin layer” as used herein refers to the three layers of the human skin, namely the epidermis, the dermis, and the hypodermis. The epidermis is the outermost layer of skin, provides a waterproof barrier and creates our skin tone. The average thickness of the epidermis layer is about 0.1 millimeter (mm), which is about the thickness of one sheet of paper. In the initial phase of skin cancer (e.g. melanoma), the cancer cells may spread in the superficial layer of the skin such as the epidermis, and as the skin cancer progresses, the cancer cells may start to invade and spread deeper in the skin, e.g. in the dermis or hypodermis.

The dermis is located between the hypodermis and the epidermis. It is a fibrous network of tissue containing tough connective tissue, hair follicles, and sweat glands. The dermis provides structure and resilience to the skin. While dermal thickness varies, it is on average about 2 millimeters thick.

The hypodermis is the deepest section of the skin. The hypodermis refers to the fat tissue and connective tissue below the dermis that insulates the body from cold temperatures and provides shock absorption. Fat cells of the hypodermis also store nutrients and energy. The hypodermis is the thickest in the buttocks, palms of the hands, and soles of the feet. As we age, the hypodermis begins to atrophy, contributing to the thinning of aging skin.

The term “removing interstitial fluid” (any amount thereof) as used herein refers to using a suitable microneedle device to remove or extract (e.g. via the action of a pump or capillary forces) an amount of (aqueous) fluid surrounding the cells, for example fluid that fills the spaces between the cells e.g. to remove or extract interstitial fluid out of the skin tumor or its microenvironment, i.e. the interstitial fluid may be taken from inside (within) the skin tumor and/or around the skin tumor, by using the method as taught herein. It is understood that the microneedle device may be adjusted to penetrate the skin to a given depth depending on the tumor's size and stage (or degree of invasiveness into the skin layers). For instance, as a skin tumor becomes more invasive, the skin tumor cells (e.g. melanoma cells) begin to cross the basement membrane of the epidermis and enter the dermis, where they further proliferate and settle. Therefore, depending on the stage or invasiveness of the skin tumor, the microneedle device may be adjusted to reach a depth ranging between about 0.05 and 1.5 mm, such as for instance between about 0.1 to 1.4 mm, between about 0.2 to 1.3 mm, between about 0.3 and 1.2 mm, between about 0.4 and 1.1 mm, between about 0.5 and 1.0 mm, or between about 0.6 to 0.900 mm, or preferably between about 0.3 and 0.700 mm. It is understood that the depth may be adjusted depending on the case at hand (type of cancer, stage of disease, size of cancer, etc.), as explained above.

The term “immune checkpoint blockade inhibitor(s)” as used herein refers to a compound(s) or pharmaceutical agent(s) or drug(s) or candidate drug(s) (e.g. antibodies, fusion proteins, small molecule drugs (natural or synthetic), interfering RNA (e.g. siRNA) that totally or partially reduces, inhibits, interferes with or modulates one or more immune checkpoint proteins or their ligands, particularly inhibitory immune checkpoint molecules such as PD-1 or CTLA-4 and/or the PD-1 ligand PD-L1.

Non-limiting examples of PD-1 inhibitor compounds include PD-1 antibodies such as nivolumab (Opdivo®, Bristol-Myers Squibb), pembrolizumab (Keytruda®, Merck), BGB-A317, and others such as PDR001 (Novartis). Further PD-1 inhibitors also include any anti-PD-1 antibody described in U.S. Pat. Nos. 8,008,449, 7,521,051 and 8,354,509. Also contemplated are fusion proteins that bind to PD-1 (e.g. anti-PD-1 fusion proteins AMP-224 (MedImmune) and AMP-514 (MedImmune)).

Non-limiting examples of PD-L1 inhibitor compounds include anti-PD-L1 antibodies such as durvalumab (MED14736, Imfinzi®, MedImmune), atezolizumab (Tecentriq®, Roche), avelumab (Bavencio®, Merck), and others such as BMS-936559 (BMS) (Meng et al (2015), Cancer Treatment Review, Vol. 41, pages 868-876; Brahmer et al (2010) J Clin Oncol 28:3167-75; Brahmer et al (2012) N. Engl. J. Med. Vol: 366, pages 2455-65; Flies et al (2011) Yale J. Biol. Med. Vol. 84, pages 409-21; Topalian et al. (2012b) N. Engl. J. Med. Vol. 366, pages 2443-54; Diggs et al (2017), Biomarker Research, Vol. 5:12, pages 1-6). Further PD-L1 inhibitors include any anti-PD-L1 antibody described in U.S. Pat. No. 8,383,796. Also contemplated are fusion proteins that bind to PD-L1.

Non-limiting examples of CTLA-4 inhibitor compounds include ipilimumab ((Yervoy®, MDX-010, Bristol-Myers Squibb, FDA approved for melanoma in 2011) and (not yet approved) is tremelimumab (CP-675206, Pfizer) (Postow et al (2015) J. Clinical oncology, Vol. 33, pages 1974-1983; Pardoll, D. et al (2012), Nature Reviews Cancer, Vol. 12, pages 252-264).

The term “immune checkpoint molecule” as used herein refers to a protein that is expressed by T cells or other immune cells that either turn up a signal (also known as “stimulatory checkpoint molecules”) or turn down a signal (also known as “inhibitory checkpoint molecules”). Five stimulatory checkpoint molecules on T cells are members of the tumor necrosis factor (TNF) receptor superfamily—CD27, CD40, OX40, GITR and CD137. Another two stimulatory checkpoint molecules belong to the B7-CD28 superfamily—CD28 itself and ICOS. Inhibitory checkpoint molecules have been increasingly considered as new targets for cancer immunotherapy due to their potential for use in multiple types of cancers. Currently approved checkpoint inhibitors block CTLA-4 and PD-1, and also one of the ligands of PD-1, PD-L1. In the context of the present invention, PD-1 and PD-L1 are particularly preferred, alone or in combination with other immune checkpoint therapy agents such as CTLA-4 inhibitor compounds.

The term “chemotherapeutic agent” as used herein refers to any chemical agent with therapeutic usefulness in the treatment of diseases characterized by abnormal cell growth. Such diseases include tumors, neoplasms, and cancer. Non-limiting examples of chemotherapeutic agents suitable for treating melanoma include those known by those skilled in the art for treating a skin tumor, such as melanoma, including Adriamycin (doxorubicin), Dacarbazine, Temozolomide, Paclitaxel, Cisplatin, Carboplatin, Vinblastine, and others. Non-limiting examples of chemotherapeutic agents suitable for treating basal-cell skin cancer or squamous-cell skin cancer (SCC) include Cisplatin, Adriamycin, Adrucil (5-fluorouracil (5-FU)), Capecitabine, Topotecan, Etoposide, and others.

The present inventor has found a new method for treating cancer, particularly skin cancer, such basal-cell skin carcinoma, squamous-cell skin carcinoma, and melanoma. More specifically, the present inventor found that by removing an amount of interstitial fluid from within the skin tumor, with or without the interstitial fluid from the surrounding of a skin tumor (e.g. melanoma), by using the method as taught herein, several beneficial effects are observed including: 1) impairment of tumor growth, 2) impairment of tumor's ability to undergo metastasis or spread to other parts of the body, and 3) improvement of host immunity (e.g. host's immune cells can better infiltrate the tumor, detect and eliminate (kill) cancer cells). Globally, the beneficial effects can result in reduced tumor volume and increased survival over time.

In addition, removal of an amount of interstitial fluid from within and/or from the surrounding of a skin tumor, in a subject undergoing a drug treatment (e.g. oral or systemic administration of a cancer therapeutic such as an immune checkpoint blockade inhibitor, a chemotherapeutic, etc.), may enhances the effect of the drug treatment (compared to drug treatment alone).

Without being bound to any theories, it is believed that the beneficial effects associated with the method as taught herein occur because the removal of an amount of interstitial fluid from within and/or from the surrounding of a tumor leads to:
1) a temporary void (relatively empty or emptier space), which is (at least partially) refilled with healthy interstitial fluid originating from neighboring healthy cells. As evidenced by the (in vitro) experimental data provided herein, exposing cells (e.g. keratinocytes cells (HaCaT cells) or endothelial cells) to a heathy fluid (cell culture supernatant obtained from the same cells cultured in normal (healthy) conditions, mimicking healthy interstitial fluid) or normal exosomes purified from the same healthy supernatant, protects said cells from further inflammatory challenge with TNF alpha. It is believed that the same phenomenon occurs in vivo, i.e. the healthy cells within the skin tumor environment will be protected from the detrimental influence of cancer cells and secretions thereof (e.g. immunosuppressive cytokines, chemokines and tumor-derived EVs, hypoxic microenvironment) as a result of the refilling with healthy interstitial fluid (as explained above), and/or
2) the skin cancer microenvironment is depleted from deleterious molecules, which either counteract the effect of cancer drugs and/or contribute to the tumor's ability to growth, escape immune surveillance and/or undergo metastasis so as to spread to other parts of the body. By depleting such deleterious molecules, cancer drugs can exert their effects without interference, which lead to an increase in skin tumor cell death, and ultimately decreased skin tumor volume or disappearance of the skin tumor, and increased survival.

Prior to the filing of the present invention, the therapeutic strategies aiming at directly targeting the tumor microenvironment were limited to the checkpoint blockade inhibitors. By blocking a certain type of physical interaction between the tumor cells and the immune cells (CTLA4/CTLA4L or PD-1/PD-1L), these therapeutics only deal with one component of the tumor microenvironment: the cell-cell contact. However, the targeting of the 2 other components of the TME (the ECM and, most important, the soluble factors) remains unaddressed with this strategy.

Hundreds of different soluble factors are present in the skin microenvironment under healthy condition and can be modified in the case of a skin tumor, contributing to local immunosuppression, tumor spread and metastasis. Moreover, each tumor differs with regard to the soluble factors that are present in its microenvironment. Therefore, it is virtually impossible to target them all with the classical pharmacological approach relying on inhibition of single molecular targets by use of small molecules or antibodies.

Therefore, by physically removing the totality or at least a part of all the soluble factors present in the tumor microenvironment at once (using the method as taught herein), in a continuous and/or repetitive manner and for an extended period of time (as opposed to the extraction of single small volumes for diagnosis), the present method represents a new strategy for the treatment of skin cancer, which had not been envisaged before. The further advantages of the invention will become evident throughout the description of the various embodiments as taught herein.

In a first aspect, the present invention relates to a method for treating a subject with a skin tumor said method comprising the step of removing (an amount of) interstitial fluid from said skin tumor.

In an embodiment, the skin tumor may be any skin tumor, at any stage of development, such as melanoma or non-melanoma tumors (or cancers) (e.g. basal-cell skin carcinoma, squamous-cell skin carcinoma).

In an embodiment, the skin cancer may be selected from melanoma, basal-cell skin carcinoma, and squamous-cell skin carcinoma. In an embodiment, the skin cancer is a non-melanoma cancer such as basal-cell skin carcinoma, and squamous-cell skin carcinoma. In a preferred embodiment, the skin cancer is melanoma.

In an embodiment, the removal of interstitial fluid from the skin tumor may be performed at any stage of the skin cancer, however ideally (preferably) it is performed before the cancer (e.g. melanoma) has metastasized (has spread to other area(s) of the body).

In an embodiment, the removal of interstitial fluid from the skin tumor is performed by using a microneedle device.

In an embodiment, the microneedle device for the removal of interstitial fluid from the skin tumor comprises:
a plurality of microneedles each having a base end and a tip;
a substrate to which the base ends of the microneedles are attached or integrated;
a reservoir for receiving interstitial fluid, said reservoir being operationally connected to the plurality of microneedles;
a pump for removing interstitial fluid, said pump being operationally connected to the plurality of microneedles; and
optionally, an adhesive means.

In an embodiment, any microneedle device suitable for removing (an amount of) interstitial fluid from a skin tumor, according to the method as taught herein and fulfilling the criteria above, may be used. Non-limiting examples of suitable microneedle devices include the device described in US2016296149 or the microneedle device described in Mukerjee et al (2004), Sensors and Actuators A., Vol. 114, pages 267-275).

In an embodiment relating to the method as taught herein, the interstitial fluid may be removed from the skin tumor in a sustained or intermittent manner, at a rate ranging between about 50 to 1500 microliters per day (24 hours), for example depending on the tumor volume, such as for instance, preferably between about 75 to 100 microliters per day.

In an embodiment, the amount of interstitial fluid removed over a period of 24 hours may range from between about 50 to 1500 microliters per day (24 hours), such as for instance between about 55 to 1000 microliters per day, between about 60 to 500 microliters per day, between about 70 to 250 microliters per day, between about 80 to 200 microliters per day, between about 90 to 150 microliters per day, preferably between about 75 to 100 microliters per day.

In an embodiment relating to the method as taught herein, the interstitial fluid may be removed from the skin tumor in a sustained or intermittent manner, at a rate of at least 50 microliters per day (24 hours), at least 75 microliters per day (24 hours), at least 100 microliters per day (24 hours), at least 250 microliters per day (24 hours), or at least 500 microliters per day (24 hours), for example, depending on the tumor volume.

In an embodiment, the amount of interstitial fluid removed over a period of 24 hours may range from between about 50 to 1500 microliters per day (24 hours), such as for instance between about 55 to 1000 microliters per day, between about 60 to 500 microliters per day, between about 70 to 250 microliters per day, between about 80 to 200 microliters per day, between about 90 to 150 microliters per day, preferably between about 75 to 100 microliters per day.

It is understood that removing interstitial fluid in a sustained manner means without interruption over a given period, e.g. 24 hours. For instance, the interstitial fluid may be continuously removed (via the pumping action of the pump) during a period of 24 hours.

It is further understood that removing interstitial fluid in an intermittent manner means that one or more interruptions are possible over a given period, e.g. 24 hours. The interstitial fluid is thus removed in separate events, for instance the interstitial fluid may be removed twice per day (2 separate events), wherein in the first event, the interstitial fluid is removed in a continuous manner for a given period of time, e.g. 12 hours, and then the removal of the interstitial fluid is interrupted (e.g. by stopping the pump or removing the microneedle device from the skin tumor). In the second event, the removal of the interstitial fluid is resumed, wherein the interstitial fluid is removed in a continuous manner for a given period of time, e.g. 12 hours, and so on, i.e. the same principle applies for 3, 4, 5, 6 or more separate events spread over a period of 24 hours.

In an embodiment, removing interstitial fluid from the skin tumor or its environment, either in a continuous or intermittent manner (as explained above) may be repeated daily such as e.g. for at least 2 days, 3 days, 4 days, 5 days, 6 days, 7 days, 10 days, 15 days, 20 days, 25 days, 30 days, 35 days, 40 days, 45 days, 50 days, 55 days, 60 days or more. Treatment may, for example, be between 2-365 days, for example between 2 and 100 days.

In an embodiment, when using the microneedle device, as a stand-alone therapy, according to the method as taught herein, the frequency of interstitial fluid removal may for example range from 5 to 60 days, such as 5 days, 6 days, 7 days, 10 days, 15 days, 20 days, 25 days, 30 days, 35 days, 40 days, 45 days, 50 days, 55 days, or 60 days. It is understood that, if necessary, the microneedle device (patch) can be replaced by a new one (once or more) during the treatment.

In an embodiment, it is also possible to incorporate a so-called “treatment holiday” (i.e. a treatment break) between treatment bouts, e.g. there is no removal of interstitial fluid for 1 day, 2, days, 3 days, 4 days, 5 days, 6 days or 7 days or more, after which removal of the interstitial fluid is resumed, e.g. the interstitial fluid is removed in a continuous or intermittent manner (as explained above) over a period of 24 hours, and this can be repeated for a duration of, for example, at least two days, such as 5 to 60 days, such as at least 5 days, 6 days, 7 days, 10 days, 15 days, 20 days, 25 days, 30 days, 35 days, 40 days, 45 days, 50 days, 55 days, or 60 days.

In an embodiment, when using the microneedle device in a patient being treated with a cancer therapeutics (e.g. administered orally or systematically), according to the method as taught herein, the frequency of interstitial fluid removal may be at least 2 days, such as for instance at least 2 days, 3 days, 4 days, 5 days, 6 days, 7 days, 10 days, 15 days, 20 days, 25 days, 30 days, 35 days, 40 days, 45 days, 50 days, 55 days, 60 days or more. It is understood, that depending on the cancer therapeutic used, the frequency of interstitial fluid removal may be adapted to match the cancer therapeutic regimen, although not essential. For instance, checkpoint blockade inhibitors are infused intravenously once every 2 or 3 weeks for a period ranging from 3 months to 2 years. Therefore, the frequency of interstitial fluid removal may be matched to this particular regimen. It is also possible to follow the frequency of interstitial fluid removal as described above, regardless of the cancer therapeutic regimen. It is also possible to initiate the removal of the interstitial fluid, according to the frequency taught above, prior initiating a treatment with a cancer therapeutic (e.g. chemotherapy, checkpoint blockade inhibitors, etc.), such as starting to remove interstitial fluid 1 day, 2 days, 3 days, 4 days, 5 days, or more prior treatment with a cancer therapeutic is initiated in the same patient. The skilled person is aware of the possible treatment modalities to treat skin cancer, and can select an appropriate course of action with respect to frequency of removal of interstitial fluid.

In an embodiment, the method further comprises treatment with a therapeutic agent selected from the group of an immune checkpoint blockade inhibitor, an adjuvant agent, an oncolytic virus, an engineered T-cell, an engineered dendritic cell, a vaccine, a BRAF inhibitor, a MEK inhibitor, and a chemotherapeutic agent, and combination thereof. Therefore, this can be viewed as a combination treatment comprising removing of the interstitial fluid as taught herein while administering a (systematic) treatment using a therapeutic agent or drug listed above.

The term “therapeutic agent(s)” as used herein refers to an agent(s) (e.g. drug(s)) used in the treatment of a disease, such as skin cancer (e.g. melanoma)). In the context of the present invention, the therapeutic agent(s) or drug(s) is selected from an immune checkpoint blockade inhibitor, an oncolytic virus, an engineered immune cell (such as an engineered T-cell or an engineered dendritic cell), a (cancer) vaccine, a BRAF inhibitor, a MEK inhibitor, and a chemotherapeutic agent, and any combination thereof.

In an embodiment, the immune checkpoint blockade inhibitor (as defined herein) may be any suitable (for use in the method as taught herein) immune checkpoint blockade inhibitors. In an embodiment, the immune checkpoint blockade inhibitor is selected from the group of ipilimumab, nivolumab, pembrolizumab, AMP-224 AMP-514, durvalumab, atezolizumab, avelumab, BMS-936559, and tremelimumab, preferably ipilimumab, nivolumab, and pembrolizumab or any combination thereof, more preferably ipilimumab.

The term “oncolytic virus” as used herein refers to a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses. In an embodiment, the oncolytic virus may be any suitable oncolytic virus. In an embodiment, the oncolytic virus may be selected from the group of Imlygic (also known as talimogene laherparepvec) obtainable at Amgen (Thousand Oaks, Calif., USA) and HF-10 obtainable at Takara Bio (Shiga, Japan), and any combination thereof.

The term “engineered immune cell” as taught herein refers to artificially modified immune cells such as T cells and dendritic cells.

The term “engineered T cell” as used herein refers to a T lymphocyte cell having a T cell receptor (TCR) at its surface, which has been engineered (artificially modified) to combine a new specificity with an immune cell to target cancer cells. Typically, the TCR is modified to graft the specificity of a monoclonal antibody onto a T cell. The engineered TCRs are often called chimeric because they are fused with an artificial component (e.g. antibody) from different sources (non-endogenous). A non-limiting example of an engineered T cell is a CAR-T cell.

In an embodiment, the engineered immune cell is an engineered T-cell (any suitable engineered T-cell).

The term “engineered dendritic cell” as used herein refers to dendritic cells (known as antigen-presenting cells), which have been artificially modified or engineered to present tumor antigens to cytotoxic T lymphocytes by loading with, or forced expression of tumor (neo)antigens. In an embodiment, the engineered immune cell is an engineered dendritic cell (any suitable engineered dendritic cell).

The term “vaccine” (or cancer vaccine) as used herein refers to a vaccine that either treats existing cancer or prevents development of a cancer. Vaccines that treat existing cancer are known as therapeutic cancer vaccines. In an embodiment, the (cancer) vaccine may be any suitable vaccine. In an embodiment, the vaccine may be selected from the group of GVAX (Aduro Biotech, Berkeley, Calif., USA), NEO-PV-01 (Neon therapeutics, Cambridge, Mass., USA), GEN-009 (Genocea, Cambridge, Mass., USA), VB10.NEO (Vaccibody, Oslo, Norway), NCI-4650 (Moderna therapeutics, Cambridge, Mass., USA), and YE-NEO-001 (NantBioSciences, Culver City, Calif. USA), and any combination thereof.

The term “BRAF inhibitor” as used herein refers to a compound or a drug that inhibits the enzyme B-Raf (protein product of the BRAF gene), which plays a role in the regulation of cell growth. BRAF is often mutated in melanoma. In an embodiment, the BRAF inhibitor may be any suitable BRAF inhibitor. In an embodiment, the BRAF inhibitor may be selected from the group of Zelboraf (also known as vemurafenib) obtainable from Genentech (San Francisco, USA) and Tafinlar (also known as dabrafenib) obtainable from Novartis (Basel Switzerland), and any combination thereof.

The term “MEK inhibitor” as used herein refers to a compound or drug that inhibits the mitogen-activated protein kinase enzymes MEK1 and/or MEK2. MEK inhibitors can be used to affect the MAPK/ERK pathway which is often overactive in some cancers. In an embodiment, the MEK inhibitor may be any suitable MEK inhibitor. In an embodiment, the MEK inhibitor may be selected from the group of Mekinist (also known as trametinib) obtainable at Novartis (Basel, Switzerland), Cobimetinib (also known as cobimetinib) obtainable at Roche (Almere, NL), Mektovi (also known as binimetinib obtainable at Array Biopharma (Boulder, Colo., USA), and PD0325901 obtainable at Pfizer (New York, USA), and any combination thereof.

The term “chemotherapeutic agent” (known as chemo drugs) as used herein refers to a cytotoxic compound or a drug capable of inhibiting or reducing mitosis (cell division). In an embodiment, the chemotherapeutic agent may be any suitable chemotherapeutic agent. In an embodiment, the chemotherapeutic agent may be selected from Dacarbazine, Temozolomide, Nab-paclitaxel, Paclitaxel, Cisplatin, Carboplatin, and Vinblastine, and any combination thereof.

In an embodiment, the method further comprises treatment performed according to a therapeutic regimen selected from radiotherapy and laser therapy.

In a preferred embodiment, the therapeutic agent is a checkpoint blockade inhibitor selected from a PD-1 antagonist, a PD-L1 antagonist, and a CTLA-4 antagonist and/or an engineered immune cell, selected from and engineered T cell (preferably Yescarta) and an engineered dendritic cell (preferably DCP-001) (as taught herein), and any combination thereof. In a further preferred embodiment, the therapeutic agent is Nivolumab.

In an embodiment, the therapeutic agent (e.g. Nivolumab) is delivered to the skin tumor via systemic circulation, e.g. following intravenous injection with the therapeutic agent or following oral administration of the therapeutic agent, etc.

In an embodiment, the therapeutic agent may be provided (preferably by intravenous injection or oral administration) to a subject prior removing the interstitial fluid from a skin tumor in said subject or simultaneously during removal of the interstitial fluid from a skin tumor in said subject or after removing the interstitial fluid from a skin tumor in said subject or any combination thereof (e.g. before, during and after). The teaching, advantages and preferences taught above also apply here fully.

In a further aspect, the present invention relates to a therapeutic agent (as taught herein) for use in a method for treating a subject with a skin tumor (e.g. melanoma), wherein the therapeutic agent is selected from the group of an immune checkpoint blockade inhibitor, an oncolytic virus, an engineered immune cell (such as an engineered T-cell or an engineered dendritic cell), a (cancer) vaccine, a BRAF inhibitor, a MAPK inhibitor, a MEK inhibitor, and a chemotherapeutic agent, and any combination thereof, and wherein the treatment comprises removing interstitial fluid from the skin tumor or its environment (surrounding) (by the method as taught herein).The therapeutic agent may be selected as taught above.

In a further aspect, the present invention relates to an interstitial fluid obtained from a skin tumor (e.g. melanoma) for use in the treatment of said skin cancer, wherein the skin cancer is treated by removal of the interstitial fluid from the skin tumor. The teaching, advantages and preferences taught above also apply here fully.

### WORKING EXAMPLES

### Example 1

To illustrate the efficacy of the method of the invention, an in vitro model of conditioned media (CM) transfer was used.

### Materials and Method

### 1.1. Preparation of Conditioned Medium (CM)

Step 1: Two groups of confluent keratinocyte cell-line culture (HaCaT cells passage 37, purchased from ATCC Teddington, Middlesex UK) were treated with 1) the pro-inflammatory cytokine TNF alpha (Sigma-Aldrich, Zwijndrecht, NL) at 50 ng/ml (“inflamed group 1”, i.e. the test condition) or 2) with culture medium (Dulbeco's Modified Eagles Medium (DMEM) comprising 10% foetal calf serum and antibiotics) only (“healthy group 1”, i.e. the control condition). Both experimental groups were exposed to their respective treatment condition for 24 hours in a 96 well plate in a humidified incubator at 37 degree Celsius and under a 5% CO₂ atmosphere.

Step 2: At the term of the 24-hour period, the culture medium was removed in both experimental groups, and discarded.

Step 3: The keratinocyte cells in both treatment groups were washed twice with phosphate-buffered saline (PBS), after which fresh medium (100 ul) was added in the wells. The well-plates containing the cells from both experimental groups were placed back in the incubator for a period of 24 hours. This strategy (challenge with TNF alpha followed by wash with PBS and addition of fresh medium) was used to avoid any potential artefact linked to the presence of exogenous TNF alpha in the inflamed group. Further, during these periods, the cells were left undisturbed, i.e. allow to grow and produce any secretion product in the culture medium. The composition of said medium (i.e. comprising any secretion products from the cells) is referred herein as the “conditioned medium (CM)” in the inflamed group 1 while for the healthy group 1, the composition of the medium is referred to as “non-conditioned medium”.

Step 4: At the term of the 24-hour period, the CM were collected from both experimental groups and kept aside for use in a subsequent experiment (see (b) below).

### 1.2. Impact of CM on TNF Alpha-Induced Inflammatory Response

Next, the effects of the CM (generated in experiment (a)) on the TNF alpha-induced inflammatory response (as measured by interleukin 8 (IL-8) secretion) in keratinocyte cells (HaCaT cells) was assessed as follows:

Six experimental groups were used:

Group 1 (control): Confluent keratinocyte cell-line culture (HaCaT cells passage 37) maintained in the (basic) culture medium for 48 hours, with a PBS wash after the first 24 hours.

Group 2 (control): Confluent keratinocyte cell-line culture (HaCaT cells passage 37) maintained in the (basic) culture medium for 24 hours, washed with PBS and then exposed to non-conditioned medium (healthy) of experiment (1.1) for 24 hours.

Group 3 (control): Confluent keratinocyte cell-line culture (HaCaT cells passage 37) maintained in the (basic) culture medium for 24 hours, washed with PBS and then exposed to the conditioned medium (inflamed) of experiment (1.1) for 24 hours.

Group 4 (TNF alpha 50 ng/ml)): Confluent keratinocyte cell-line culture (HaCaT cells passage 37) maintained in culture medium comprising TNF alpha (50 ng/ml) for 24 hours, washed with PBS and then exposed to the (basic) culture medium for 24 hours.

Group 5 (TNF alpha 50 ng/ml)): Confluent keratinocyte cell-line culture (HaCaT cells passage 37) maintained in culture medium comprising TNF alpha (50 ng/ml) for 24 hours, washed with PBS and then exposed to the non-conditioned medium (healthy) of experiment (1.1) for 24 hours.

Group 6 (TNF alpha 50 ng/ml)): Confluent keratinocyte cell-line culture (HaCaT cells passage 37) maintained in culture medium comprising TNF alpha (50 ng/ml) for 24 hours, washed with PBS and then exposed to the conditioned medium (inflamed) of experiment (1.1) for 24 hours.

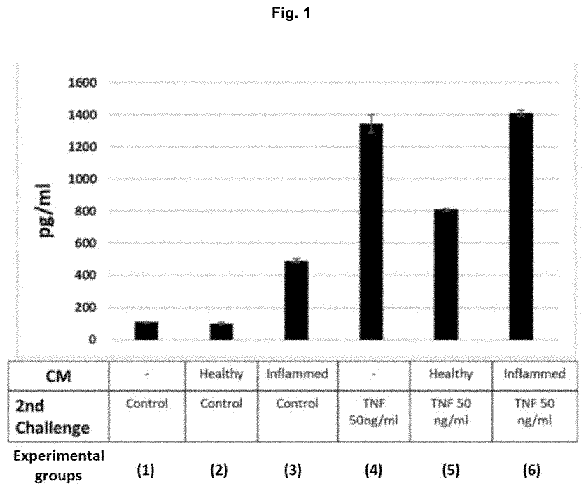

At the term of the treatment, the supernatants were collected from each experimental group and IL-8 was measured by ELISA. The results are shown in FIG. 1. Specifically, the results show that the inflamed CM transferred the inflammation to the control cells (see group 3), as evidenced by the increase in IL-8 levels compared to the control situation (see groups 1 and 2). This result was expected as it is known that CM can transfer inflammation between cells.

The results further show that healthy CM was able to protect the cells from a subsequent inflammatory challenge, as the cells treated with healthy CM showed around 40% reduction in IL-8 production in response to the TNF alpha challenge (see group 5) compared to group 6 (inflamed CM).

### Example 2

To illustrate the efficacy of the method of the invention, an in vitro model of micro vesicle transfer was used.

### 2.1 Preparation of Exosomes

Exosomes were purified using the miRCURY exosome isolation kit (Qiagen) according to manufacturer's instructions.

Normal exosomes: normal exosomes were isolated from a 1-day culture supernatant of confluent endothelial cells (primary cells Human Umbilical Vein Embryonic Cells, known as HUVEC, supplied by Lonza Basel, Switzerland) grown in a 25 cm2 flak in 5 ml of a culture medium (Dulbeco's Modified Eagles Medium (DMEM) comprising 10% FCS with no antibiotics)) that were left untreated, i.e. incubated in a culture medium for 24 hours.

Inflamed exosomes: inflamed exosomes were isolated from a 1-day culture supernatant of confluent endothelial cells (primary cells Human Umbilical Vein Embryonic Cells, known as HUVEC, supplier Lonza Basel, Switzerland) grown in a 25 cm2 flak in 5 ml of a culture medium (Dulbeco's Modified Eagles Medium (DMEM) that were treated with TNF alpha (50 ng/ml, same supplier as above), i.e. incubated in a culture medium comprising TNF alpha (50 ng/ml) for 24 hours.

### 2.2 Impact of Exosomes on TNF Alpha-Induced Inflammatory Response

Next, the effects of the exosomes (generated in experiment (a)) on the TNF alpha-induced inflammatory response (as measured by interleukin 6 (IL-6) secretion) in endothelial cells were assessed as follows:

Six experimental groups were used:

Group 1 (control): a 1-day confluent culture of endothelial cells was incubated in a culture medium for 24 hours.

Group 2: a 1-day confluent culture of endothelial cells was incubated in a culture medium for 2 hours. At the term of the 2-hour incubation, the endothelial cells were incubated in a culture medium comprising TNF alpha (50 ng/ml) for 24 hours.

Group 3: a 1-day confluent culture of endothelial cells was incubated for 2 hours in a culture medium comprising normal exosomes (as obtained in experiment 2.1) in a 1:1 surface ratio (i.e. the amount of exosomes added to each surface unit of the 1-day confluent culture corresponded to the totality of the exosomes produced by the same surface unit in experiment 2.1),. At the term of the 2-hour incubation, the endothelial cells were incubated in a culture medium for 24 hours.

Group 4: a 1-day confluent endothelial cells were incubated in a culture medium comprising normal exosomes (as obtained in experiment 2.1) in a 1:1 surface ratio, for 2 hours. At the term of the 2-hour incubation, the endothelial cells were incubated in a culture medium comprising TNF alpha (50 ng/ml) for 24 hours.

Group 5 a 1-day confluent endothelial cells were incubated in a culture medium comprising inflamed exosomes (as obtained in experiment 2.1) in a 1:1 surface ratio, for 2 hours. At the term of the 2-hour incubation, the endothelial cells were incubated in a culture medium for 24 hours.

Group 6: a 1-day confluent endothelial cells were incubated in a culture medium comprising inflamed exosomes (as obtained in experiment 2.1) in a 1:1 surface ratio, for 2hours. At the term of the 2-hour incubation, the endothelial cells were incubated in a culture medium comprising TNF alpha (50 ng/ml) for 24 hours.

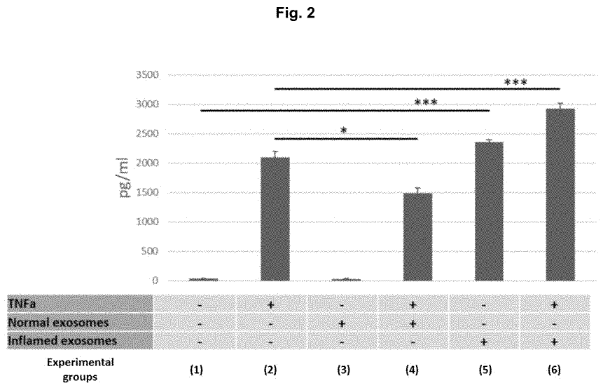

At the term of the treatment, the supernatants were collected from each experimental group and IL-6 was measured by ELISA. The results are shown in FIG. 2. Specifically, it can be observed that that inflamed exosomes were capable to transfer the inflammation to the control cells (see group 5 compared to group 1 (control)). This result was expected as it is known that exosomes can transfer inflammation between cells.

The results further show that normal exosomes were able to protect the cells from a subsequent inflammatory challenge, as the cells pre-treated with normal exosomes showed a significant 25% reduction in IL-6 production in response to a TNF alpha challenge (see group 4 compared to group 2). It is also observed that pre-treatment with inflamed exosomes worsen the outcome (more IL-6 secretion) of a TNF alpha challenge (see group 6) compared to cells exposed to TNF alpha only (see group 2).

Having now fully described this invention, it will be appreciated by those skilled in the art that the same can be performed within a wide range of equivalent parameters, concentrations, and conditions without departing from the spirit and scope of the invention and without undue experimentation.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

1. A method for treating a subject with a skin tumor said method comprising the step of removing an amount of interstitial fluid from said skin tumor.

2. The method according to claim 1, wherein the removal of interstitial fluid from the skin tumor is performed by using a microneedle device.

3. The method according to claim 2, wherein the microneedle device for the removal of interstitial fluid from the skin tumor comprises:
a plurality of microneedles each having a base end and a tip;
a substrate to which the base ends of the microneedles are attached or integrated;
a reservoir for receiving extracellular fluid and/or intracellular fluid, said reservoir being operationally connected to the plurality of microneedles;
a pump for removing extracellular fluid and/or intracellular fluid, said pump being operationally connected to the plurality of microneedles; and
optionally, an adhesive means.

4. The method according to claim 3, wherein the plurality of microneedles are of varying length.

5. The method according to claim 2, wherein the microneedle device is positioned on the skin tumor, or on skin tissue adjacent to the skin tumor or in part on the skin tumor and in part on skin adjacent to the skin tumor.

6. The method according to claim 1, wherein the interstitial fluid is removed from the skin tumor in a sustained or intermittent manner, at a rate of at least 50 microliter per day.

7. The method according to claim 1, wherein the step of removing interstitial fluid from the skin tumor is repeated daily for at least 2 days, 3 days, 4 days, 5 days, 6days, 7 days, 10 days, 15 days, 20 days, 25 days, 30 days, 35 days, 40 days, 45 days, 50 days, 55 days, 60 days or more.

8. The method according to claim 1, further comprising treatment with a therapeutic agent selected from the group of an immune checkpoint blockade inhibitor, an agent or therapeutic regimen, an oncolytic virus, an engineered T-cell or dendritic cell, a (cancer) vaccine, a BRAF inhibitor, a MAPK inhibitor, a MEK inhibitor and a chemotherapeutic.

9. The method according to claim 8, wherein the immune checkpoint inhibitor is selected from a PD-1 antagonist, PD-L1 antagonist, and/or CTLA-4 antagonist and/or an engineered immune cell, selected from and engineered T cell and an engineered dendritic cell.

10. The method according to claim 8, wherein the chemotherapeutic agent is selected from Dacarbazine, Temozolomide, Nab-paclitaxel, Paclitaxel, Cisplatin, Carboplatin, and Vinblastine.

11. A therapeutic agent for use in a method for treating a subject with a skin tumor, wherein the therapeutic agent is selected from the group of an immune checkpoint inhibitor, an oncolytic virus, an engineered T-cell, a (cancer) vaccine, a BRAF inhibitor, a MAPK inhibitor, a MEK inhibitor, and a chemotherapeutic agent, and wherein the treatment comprises removing interstitial fluid from the skin tumor.

12. An extracellular fluid and/or intracellular fluid obtained from a skin tumor for use in the treatment of skin cancer, wherein the skin cancer is treated by removal of the interstitial fluid from the skin tumor.
